(19) United States
(12) Patent Application Publication
(10) Pub. No.: US 2010/0250280 A1
(43) Pub. Date: Sep. 30, 2010

(54) Medicament Delivery Systems

(75) Inventor: Garth Campbell Sutherland, Auckland (NZ)

Correspondence Address: Davidson, Davidson & Kappel, LLC, 485 7th Avenue, 14th Floor, New York, NY, 10018, US

(73) Assignee: Nexus6 Limited, Auckland (NZ)

(21) Appl. No.: 12/648,392

(22) Filed: Dec. 29, 2009

(30) Foreign Application Priority Data
Mar. 27, 2009 (NZ) 575836

(51) Int. Cl. G06Q 10/00; A61M 15/00; G06Q 50/00; G06F 21/00

(52) U.S. Cl. 705/3; 128/203.12; 700/231; 726/4

(57) ABSTRACT

This invention relates to a system for managing data relating to patient usage of a medicament delivery device. The medicament delivery device includes: a supply of medicament, a medicament dispensing means, a data gathering means for gathering data relating to patient usage of the medicament delivery device, a data storage means for storing the data, and communication means for transmitting the data to at least one web service. The at least one web service is preferably able to share the data with at least one computer program. The at least one web service may also be adapted to receive information or data from the at least one computer program, which may be queued, stored or transmitted to the medicament delivery device.

This application claims priority to New Zealand Patent Application No. 575836, filed Mar. 27, 2009, the contents of which are hereby incorporated by reference in their entirety.

### FIELD OF THE INVENTION

This invention relates to improvements in or relating to medicament delivery systems. More specifically, the invention relates to improved systems for managing data relating to patient usage of a medicament delivery device(s). The invention may be particularly suitable for use with medicament delivery devices used for the treatment of respiratory diseases such as asthma, COPD, cystic fibrosis, and bronchiectasis. However, it is to be understood and appreciated that the invention is not to be limited to such use. For example, the invention may also be used in relation to medicament delivery devices used to treat diabetes, as well as a whole range of other diseases, afflictions, or even addictions. The prior art and possible applications of the invention, as discussed below, are therefore given by way of example only.

### BACKGROUND OF THE INVENTION

Medicament inhalers are widely used for the treatment of diseases, and especially respiratory diseases such as asthma, COPD, cystic fibrosis, and bronchiectasis. Medicament inhalers are also becoming ever-more prevalent for use with other diseases, for example diabetes.

A common type of medicament inhaler is what is known as a pressurised Metered Dose Inhaler (pMDI). Such inhalers generally comprise a medicament canister and an actuator. The medicament canister contains medicament under pressure and is designed to deliver a metered dose of medicament in the form of an aerosol spray. The actuator generally comprises a substantially L-shaped hollow tube which has a first open end adapted to receive the medicament canister, and a second open end which acts as a mouth piece. Medicament canisters for use with a pMDI generally have a spray stem extending from one end which is adapted to engage with a spray-directing element housed within the actuator, and adjacent to the mouth piece of the actuator. When the canister is pushed down into the actuator, the spray stem and spray-directing element combine to direct a metered dose of medicament out through the mouthpiece and into the mouth of the user.

Another common type of medicament inhaler is what is known as a Dry Powder Inhaler (DPI). DPI's are generally in the form of a disc or grinder which may be rotated in order to dispense a metered dose of dry powder into an appropriate receptacle or mouthpiece, from where it may then be inhaled by the user (for example, by sucking strongly on the mouthpiece of the inhaler).

Some medicament inhalers are kept on hand for use in a specific event or emergency. For example, if a person were to have a sudden asthma attack, they may reach for a medicament inhaler which contains what is generally known as a “reliever” medicament. A reliever medicament is fast acting and in most cases will relieve (or reduce the severity of) an asthma attack, almost instantaneously.

Other medicament inhalers are designed for regular use in order to prevent an event such as an asthma attack and/or to manage or control a disease such as asthma. Such inhalers are generally known as “preventers” because the regular use of such inhalers serves to prevent (or minimise the likelihood of) an asthma attack. The regular use of preventer medicament by asthma sufferers is generally effective in controlling the disease and/or preventing the vast majority of asthma attacks. Commonly, preventer medicament for asthma sufferers is taken twice a day, usually at a set time in the morning and in the evening.

There are now also available “combination” medicament inhalers which combine both a reliever and preventer medicament, with a view to controlling the respiratory disease (with the preventer medicament), when a patient uses their reliever medicament for symptom alleviation.

Studies have shown that many people demonstrate poor disease management, for example by overusing their reliever medicament. The overuse of a reliever medicament has the potential to reduce the effectiveness of the medicament which may render the medicament less effective in times of real need, for example during a severe asthma attack.

Moreover, a patient's increased use of reliever medicament over a period of time may be indicative of a pending exacerbation event.

A problem or difficulty associated with the use of preventer (or “combination”) medicament inhalers is poor medicament compliance. That is, many studies have shown that users frequently do not take their medicament at the predetermined or prescribed times and/or in the required amounts.

The consequences of this non-compliance are reduced disease control, lower quality of life, lost productivity, hospitalisation and avoidable deaths.

Furthermore, during clinical trials (for example, to test a new preventer medicament), it is important (for the trial to be successful) that the patients in the trial take their medicament at the prescribed times and/or in the prescribed amounts.

Not only is compliance to preventative medicaments typically low, but it has also been shown that actual compliance by a user is lower than the same user's estimated compliance.

To order to address these problems and difficulties, there are available a number of compliance monitoring devices for use with medicament inhalers.

Presently available electronic compliance monitors generally include electronic dose counting means. For example, see U.S. Pat. No. 5,544,647 (Jewettt et al), U.S. Pat. No. 6,202,642 (McKinnon et al) and US Patent Publication No. 2005/0028815 (Deaton et al).

Furthermore, presently available electronic compliance monitoring devices also include means to record a range of compliance data, in addition to dose counting. For example, McKinnon includes an electronics module to record date and time as well as more comprehensive patient usage data.

U.S. Pat. No. 5,363,842 (Mishelevich et al) describes a device which also monitors patient inhalation data, for example how much air is inhaled through the inhaler and with what time course. The resultant data may be transmitted to a remote location such as a health care professional where the inhalation data can be compared to a standard target envelope and, based on the comparison, the success or failure of the patient to inhale the medicament effectively may be determined. The success or otherwise of the patent's inhalation may then be signaled back to the patient. Mishelevich also monitors for other patient usage data such as whether the medicament inhaler was shaken prior to use.

The compliance data gathered by such electronic compliance monitoring devices may be managed or used in various ways.

For example, Deaton displays the compliance data (in the form of a counter module) on a display (20) which is integrally formed with the device. This has limitations in that the compliance data is only able to be viewed by the user, whereas it would be of more benefit to have the data viewed and/or monitored by a third party such as a care giver, insurer, or a medical professional.

Jewett includes both an LCD display (54) for displaying compliance data on the device, as well as a memory for storing data which may later be downloaded to a printer via a terminal (58). McKinnon describes a docking station (300) which is adapted to receive the compliance monitoring device and retrieve the compliance data from the device, where it may be viewed and manipulated by a computer.

A disadvantage associated with Jewett, Deaton and McKinnon is that the compliance data is not able to be transmitted wirelessly and/or in real time. Instead, the compliance data is only able to be downloaded periodically, and at the sole discretion of the user. There is also no provision for the two-way transmission of data.

An advantage of monitoring patient compliance in real time (or at frequent time intervals) is that patient usage data is always current, and hence anyone viewing or wishing to have access to the data will feel more comfort knowing this. Furthermore, any potential overuse or underuse of the inhaler may be immediately apparent, and (for example) any appropriate alerts may be made to the patient (or to a third party such as a care giver or medical professional). Real time monitoring may also be able to predict a potential exacerbation event, prior to the event occurring.

An advantage associated with the wireless transmission of data, is that the data may be transferred to a remote monitoring location, for example via a mobile phone network to the internet and/or a network computer system. The remote monitoring location may be, for example, a medical professional whereby the medical professional may monitor patient usage or compliance data.

U.S. Pat. No. 6,958,691 (Anderson et al) describes such a system, namely a device for the delivery of medicament which includes a medicament inhaler with an electronic data management system. The Anderson device includes an inbuilt communicator which provides for two-way wireless communication between the electronic data management system and a network computer system. The communication may be in real time. The network computer system is integrated with a host of websites, for example those of a pharmacy and weather station.

However, a disadvantage associated with Anderson (and also to a certain extent, Mishelevich) is that such systems generally use proprietary software, which therefore has limitations as to use and applicability. Furthermore, whilst the patient data may be made available, for example, on a central database which may be accessible by authorised users, any data or analysis of the data may only be viewable on a computer connected to the network and/or within a web browser. Moreover, there is no provision for a third party's software programs to be able to obtain and integrate patient usage information into their own software programs, for example a health information system or clinical trials application.

### OBJECT OF THE INVENTION

It is an object of the present invention to provide an improved medicament delivery system which goes some way towards addressing the aforementioned problems or difficulties, or which at the very least provides the public with a useful choice.

### DEFINITIONS

Throughout this specification unless the text requires otherwise, the word ‘comprise’ and variations such as ‘comprising’ or ‘comprises’ will be understood to imply the inclusion of a stated integer or step or group of integers or steps but not the exclusion of any other integer or step or group of integers or steps.

Throughout this specification, the term “patient” when used in relation to a medicament delivery device, is to be understood to refer to any person that uses a medicament delivery device.

### SUMMARY OF THE INVENTION

According to one aspect of the present invention, there is provided a system for managing data relating to patient usage of a medicament delivery device, said medicament delivery device including:
a) a supply of medicament,
b) a medicament dispensing means,
c) data gathering means for gathering data relating to patient usage of said medicament delivery device,
d) a data storage means for storing said data, and
e) communication means for transmitting said data to at least one web service,
and wherein said at least one web service is able to share said data with at least one computer program.

### BRIEF DESCRIPTION OF THE DRAWINGS

FIG. 1: is a front perspective view of one possible embodiment of a medicament delivery device for use with the present invention,

FIG. 2: is a simplified system block diagram representing one possible embodiment of a system for managing data relating to patient usage of the medicament delivery device shown in FIG. 1,

FIG. 3: is an expanded view of part of the system block diagram shown in FIG. 2, and

FIG. 4: is a further simplified representation of the system for managing data relating to patient usage of a plurality of medicament deliver devices.

### DETAILED DESCRIPTION OF THE INVENTION

The “data” (or variations such as “dataset”) relating to the patient usage of the medicament delivery device may include (but is not limited to) any or all data or information relating to the patient; the environment (eg, temperature or humidity); the medicament; the delivery of the medicament; the quantity of medicament delivered; patient compliance data; the medicament delivery device; any physiological criteria of a patient (eg, peak flow data, inhalation data or blood glucose level data); any programs or algorithms associated with the system; any data or instructions sent or received by any component of the system; the results of any operations on any of the aforesaid data.

The use of medicament delivery devices with compliance monitoring technology such as data gathering means and/or data storage means and/or wireless communicators are known in the prior art and to those skilled in the art (eg, the prior art patents referred to in the discussion under “Background Art”). It is not intended therefore (or considered necessary) to include an overly detailed description of such compliance monitoring technology per se.

Preferably, the system provides for the remote monitoring of a plurality of medicament delivery devices, although the monitoring of a single medicament delivery device is still within the scope of the invention. It is envisaged however that the invention may be particularly useful for monitoring patient usage data in relation to a large number of medicament delivery devices, such as are used for clinical trials and the like. The number of medicament devices associated with the system may therefore run into the hundreds, if not thousands. For convenience however, the medicament delivery device will be predominantly referred to herein in the singular.

Any type of medicament delivery device is within the scope of this invention. Examples of medicament delivery devices include (but are not limited to): medicament inhalers; medicament devices for delivering a dose of insulin; nasal sprays; nebulisers; transdermal devices; and pill boxes or containers (eg those which have a compartment for medicaments to be taken each day of the week).

Any type of medicament may be utilised, as required or as desired or as dictated by the nature of the medicament delivery devices and/or the disease being treated.

Any suitable medicament dispensing means may be utilised which enables the patient to receive a dose of medicament.

The medicament delivery device may preferably be adapted to deliver a metered dose of medicament, although the delivery of a non-metered dose of medicament is also within the scope of the invention.

Preferably, the medicament delivery device may be a medicament inhaler, such as a pMDI or a DPI, and preferably an integrated electronic medicament inhaler.

The medicament delivery device may preferably include a user interface which allows the patient to access data recorded or received by the medicament delivery device, and also to change the settings of the device (for example, date/time settings and/or visual/audible alarm and notification settings). The user interface may include operational buttons and a LCD screen. Such user interfaces are known in the prior art (eg Anderson and Mishelevich).

The medicament delivery device may preferably include a power management system, for example a battery. The battery may be a replaceable battery or a rechargeable battery.

The data gathering means may include any suitable means to monitor and/or gather the data relating to patient usage of the medicament delivery device, as defined previously.

Preferably, the medicament delivery device includes, at the very least, data gathering means in the form of a dose counter and a real time clock (which may record the date/time of each delivery of a dose of medicament).

The data gathering means may preferably include an electronic dose counter.

The data gathering means may also include means for monitoring for patient usage (or compliance) data such as inhalation data; peak flow data; blood/glucose levels; quantity of medicament dispensed; environmental conditions such as temperature and humidity; whether the medicament device was shaken prior to use; as well as data relating to the patient or the medicament device itself.

The medicament delivery device may preferably include a controller for controlling the operation of the medicament delivery device and/or for performing operation(s) on the data. Such controllers are known in the prior art (eg McKinnon and Anderson).

For example, the controller may serve to manage the settings or operational configurations of the medicament delivery device such as date/time and/or the timing of any alarms or notifications. The controller may also have the ability to increase or decrease the amount of medicament dispensed by the medicament delivery device, for example the quantity of medicament dispensed per dose. The controller may do this by sending a signal to the dose dispensing means to alter the quantity of medicament dispensed per dose. The controller may also have the ability to prevent the medicament dispensing means from dispensing a dose(s) of medicament for a predetermined time period (this may be particularly important for dispensing pain medicament—where the nature of the medicament often makes the patient susceptible to taking too much medicament or possibly even becoming addicted to the medicament). Preferably any such instructions for the controller to increase/decrease the quantity of medicament dispensed, or to cease medicament dispensing for a time period, may come from a third party, via the at least one web service.

The controller may preferably include a micro processor for performing operations on the data gathered. Such micro processors for use with medicament delivery devices are known in the prior art (eg Mishelevich and Anderson).

As part of its make up, the controller may include a microcontroller and/or an Application Specific Integrated Circuit (ASIC) and/or a set of digital logic gates.

In one embodiment, the data gathering means may be a stand alone component, which gathers data relating to patient usage of the medicament delivery device, and which is adapted or able to forward the data thus gathered to the data storage means.

Alternatively, the data gathering means may comprise part of the controller.

The real time clock may be incorporated within the data gathering means or alternatively it may comprise part of the controller.

The medicament delivery device may preferably include data storage means for the storage of data relating to patient usage of the medicament delivery device. For example, the data storage means may be memory in the form of RAM or ROM.

The data storage means may also be able to store data resulting from any operations performed on the patient usage data, for example by the microprocessor. The data storage means may also be able to store data relating to any instructions or data received from the at least one web service, for example when instructions were received for the controller to limit or increase the amount of medicament to be dispensed.

The communication means may preferably be adapted or able to facilitate the wireless transmission of data from the medicament delivery device to the one or more web services.

Preferably, the communication means may use cellular communications, for example in the form a cellular chip embedded in the medicament delivery device.

The communication means may be adapted to transmit the data in real time, or preferably, the communication means may be adapted to transmit the data at pre determined intervals, for example one or twice a day.

Preferably the at least one web service includes a communications web service for communicating with, or providing the communications interface with, the communications means associated with the medicament delivery device.

The data storage means associated with the medicament delivery device may also be able to store data relating to the nature and timings of any transmissions of data from the medicament delivery device to the at least one web service, including data relating to signal strengths.

Preferably, the at least one web service further includes a data storage web service for receiving and storing the data gathered and/or transmitted by the medicament delivery device, and including the results of any operation(s) on the data. The data storage web service may also store all incoming data or information or requests (including timings and the nature of the incoming data), for example from the at least one computer program.

The data storage web service may comprise part of the at least one web service, or alternatively the data storage web service may be in communication with the at least one web service, whereby data may be readily retrieved or sent as or when required.

In one embodiment, the data storage means associated with the medicament delivery device may be adapted to retain a copy of any data transmitted to the at least one web service. This may be advantageous in that it ensures a copy of the data is retained if, for example, the data was lost or corrupted in transit to the at least one web service. The copy of the data retained by the data storage means may thereafter be deleted after a set period of time or by manual operation of the device—or by instructions received from the at least one web service.

In another embodiment, the data storage means associated with the medicament delivery device may be adapted to transmit any data to the at least one web service, without retaining a copy of the data in the data storage means (but preferably the data may only be deleted from the data storage means once a signal has been received back from the at least one web service indicating the data transfer was successful).

The data to be gathered by, and/or transmitted from, the medicament delivery device to the at least one web service, may include data relating to settings of the medicament delivery device and/or data relating to a patient's prescribed treatment regimen and/or data relating to diagnostic information concerning operation of the medicament delivery device. Some of this data may be entered into the medicament delivery device by the patient, for example by use of the user interface or a PC which is operatively connected to the device (for example via the at least one web service).

Examples of data to be gathered and/or transmitted by the medicament delivery device include (but are not limited to):
1. Personal information such as patient gender, age, location, type of condition that the patient is suffering from, prescribed treatment regimen (eg, number of doses per day and when they are to be taken), type of medicament, type of medicament delivery device, any allergies, and so on.
2 Inhalation data relating to the patient (if, for example, the medicament delivery device is a medicament inhaler). Examples of inhalation data include peak flow data, and nature, strength or effectiveness of the patient's attempt to inhale the medicament. Inhalation data may also include exhalation data, for example to diagnose a situation where the patient inadvertently exhales just prior to inhaling (which would be of particular concern for DPI's).
3. Data relating to audio and/or visual reminder settings associated with said medicament delivery device. For example, the patient may program the medicament delivery device to sound audio and/or visual alarms 2 hours after the prescribed time of taking a dose of medicament has expired.
4. Data relating to battery power levels associated with the medicament delivery device.
5. Data relating to the quantity of medicament remaining in the medicament delivery device (or supply of medicament such as a medicament container).
6. Data relating to the quantity of medicament taken.
7. Diagnostic data relating to operation of the medicament delivery device, for example, any error codes associated with the operation of the device or when the mouthpiece was last replaced or if the medicament delivery device is becoming clogged with medicament, and so on.
8. Data relating to when the supply of medicament was last changed in the medicament delivery device.
9. Data relating to any attempts to transmit data from the data storage means to the at least one web service and/or communication signal strengths between the medicament delivery device and the at least one web service.
10. Data relating to environmental conditions such as temperature or humidity or pollution levels or pollen counts.
11. Data relating to patient quality of life. For example, the patient may enter into the medicament delivery device that the patient has been suffering from night waking or insomnia. Moreover, the patient may be sent (by the at least one web service) a patient questionnaire asking for answers to a series of questions once such a condition has been diagnosed or reported. Such a questionnaire may be sent manually or automatically, for example by the at least one web service.

Preferably the at least one web service can receive, queue and/or store information or data received from the at least one computer program. Such information or data may, for example, be destined for immediate or scheduled transmission to the medicament delivery device.

Preferably, such information is received, queued and/or stored by the data storage web service.

The information received from the at least one computer program may, for example, include information or data relating to settings of the medicament delivery device and/or data relating to a patient's prescribed treatment regimen and/or data relating to the operation of the medicament delivery device.

This information or data may be provided by any party, for example the patient, a care giver, a health professional or clinical trial investigator or insurer, and so on. Some of the information may relate to a patient's desire to change the settings of the device and this may be done more easily from a PC (with which people are generally very familiar with) rather than using the user interface associated with the medicament delivery device (which the patient may find unfamiliar and/or tricky to use given its small size and generally limited function).

Examples of such data or information may include (but is not limited to):
1. Audio and/or visual reminder signals for the medicament delivery device. For example, the patient (or care giver etc) may wish to change these settings based on usage data which may result in a changed treatment regimen.
2. Information relating to dose dispensing configuration settings. For example, the quantity of medicament to be dispensed and the frequency.
3. Information about the medicament. For example, this may be sent by a health professional to the patient.
4. Information which comprises educational material for the benefit of the patient. For example, this information may include an explanation of the side effects of any medicament for patients which have shown concern in relation to using any particular type of medicament. Such information may be targeted to specific patients for particular reasons as stated. This type of information may also include regular updates, and may obviate the need for regular mail outs of information to patients which has cost saving advantages (as well as timing advantages in that the information may be received immediately by the patient after being sent electronically).
5. Instructions to stop delivery of the medicament for a specified time period. For example, if the patient usage data indicates that the patient is overusing a medicament (or if the patient has not paid for the medicament), then instructions may be sent to the medicament delivery device to stop dispensing the medicament for a predetermined time period.
6. Instructions to alter the quantity of medicament delivered by the medicament dispensing means. For example, these instructions may be received by the controller which is then able to alter the quantity of medicament to be dispensed.
7. Information relating to date and time information. For example, the date and time associated with the real time clock may be automatically updated from time to time—similar to how a computer's times are constantly updated automatically.
8. Information relating to patient location. For example, if a manufacturer of medicament delivery devices was shipping devices to many different countries for a world wide clinical trial, then instructions could be sent to remotely to upload the time and date of each country into each medicament delivery device.
9. Information relating to data transmission configuration settings. For example, changes to when data is transmitted from the medicament delivery device to the at least one web service could be made.
10. Information including software for execution on the medicament delivery device. For example, anti-virus software updates could be sent, or software to fix a bug diagnosed within the medicament delivery device, or general software improvements or updates (similar to how Microsoft regularly sends out updates to people using its software applications).
11. Information which includes user interface graphics and/or strings. For example, a new user interface structure could be sent to a medicament delivery device (for example, with larger fonts making the LCD screen easier to read for people that request it).
12. Information which includes different language settings. For example, data enabling the user interface to read in Japanese or Spanish rather than English.
13. Information which includes images. For example, the patient may wish to customise their medicament delivery device by uploading a personal photo to the LCD screen.
14. Information which includes audio files. For example, the patient may wish to customise their medicament delivery device by uploading a personal song or tune to use as a reminder ringtone.
15. Information which includes moving picture files. For example, the patient may wish to customise their medicament delivery device by uploading a personal movie to the LCD screen. Alternatively a health care professional may wish to forward an educational video.

Preferably, the at least one web service is be able to perform processes and/or computations upon the data, in order to produce a report, with the report being deliverable to the at least one computer program upon request. The report may include the data in a dataset pertaining to the requested area of interest.

Any type of processes or computations upon the data may be carried out, as required or as desired, or as dictated by the type of patient compliance monitoring and/or research being undertaken. Examples of the type of processes and/or computations undertaken may include (but are not limited to) the following:
1. A dataset which includes details of patients and/or medicament delivery devices, and/or medicaments associated with each patient.
2. A dataset which includes the dates and times medicament was dispensed by the medicament delivery device, and the time zone the medicament delivery device was in at the time of medicament delivery.
3. A dataset which includes the quantity of medicament dispensed by the medicament delivery device at the dates and times.
4. A dataset which includes patient medicament compliance to a prescribed treatment regime. For example, the dataset may include the average patient medicament compliance to a prescribed treatment regime across a patient group.
5. A dataset which includes a list of all patients who have a compliance level below and/or above a specified limit.
6. A dataset which includes a list of patients who are over using their medicament based on a predetermined limit.
7. A dataset which includes a list of all patients who are dose dumping. For example, during a clinical trial a patient may not use as much medicament as has been prescribed for them, and at the end of the trial the patient may empty their supply of medicament over a very brief period of time so that it appeared they had in fact used the correct amount of medicament. It is important that a clinical researcher be aware of this so that the results of the trial are not corrupted by such incorrect use.
8. A dataset which includes the date and time of data transmission attempts and/or communication signal strengths.
9. A dataset which includes medicament delivery device battery energy levels and/or voltage information.
10. A dataset which includes medicament delivery device diagnostic information. For example any error codes associated with the medicament delivery device over a given time period. The diagnostic information may also include device communication system attributes over a given time period, for example whether data is uploaded daily or weekly or after a certain number of doses of medicament has been dispensed.
11. A dataset which includes the amount of medicament remaining in the medicament delivery device.
12. A dataset which includes when a supply of medicament was replaced in the medicament delivery device.
13. A dataset which includes a list of patients who have dispensed medicament from their medicament delivery device at times of the day that are indicative of night waking due to sub optimal disease control.
14. A dataset which includes data relating to patient inhalation (as described previously).
15. A dataset which includes data relating to patient quality of life (as described previously).
16. A dataset which includes data relating to changes in medicament dose strength. For example those patients which have increased or decreased the amount of medicament dispensed per dose.

Preferably, the system may include a web service(s) that enables another computer program to add and/or edit and/or delete data stored within the data storage web service. Examples of the type of additions and/or edits and/or deletions that may be provided for may include (but are not limited to) the following:
1. Access privileges to information relating to a patient. For example, the ability of certain people to be able to access data relating to a particular patient may be added, edited or deleted.
2. Information concerning the type of medicament delivery device. For example, if the patient changes to a different medicament delivery device then this information may be updated to the data storage web service.
3. Information relating to a time zone associated with said medicament delivery device. For example, if the patient moves to a different country, then this information may be updated to the data storage web service.
4. Information relating to the medicament. For example, if the patient changes to a different medicament then this information may be updated to the data storage web service.
5. Information relating to a prescribed treatment regime for a medicament. For example, if the patient changes to a different treatment regime (eg, from 4 dose per week to 2 doses per day), then this information may be updated to the data storage web service.
6. Information relating to a relationship between the medicament and the medicament delivery device.
7. Information relating to a relationship between the medicament delivery device and a patient. For example, the patient may be prescribed an additional medicament delivery device to use in addition to the device already being used and this information may be uploaded to the data storage web service.
8. Information relating to when a patient started and stopped using a medicament delivery device.
9. Any information relating to a make up or members of a particular patient group.
10. Information relating to any relationship(s) between a patient and a group of patients. For example, a patient may move from a control group in a clinical trial to an active group, and this information may be uploaded to the data storage web service.
11. Information relating to a health care professional, for example details of a health care professional responsible for a number of patients or a patient group.
12. Information relating to any relationship between a patient and a health care professional.
13. Information relating to a patient's care giver.
14. Information relating to any relationship between a patient and a patient's care giver.
15. Information relating to a clinical researcher.
16. Information relating to any relationship between a patient and a clinical researcher.

Preferably, the system may include one or more system administration web services which may be able to create, update and/or remove accounts for the at least one computer program which accesses and/or utilizes the at least one web service.

In such an embodiment, the system may include one or more administration web services that transact payment and billing services in exchange for access to the at least one web service by the least one computer program.

For example, the system may provide for payment and billing services which transact on a per use basis.

Alternatively, or additionally, the system may provide for payment and billing services which transact on a subscription basis

Alternatively, or additionally, the system may provide for payment and billing services which transact a pre-paid basis

Furthermore, the payment and billing services may transact a setup fee prior to enabling access to the at least one web service by the least one computer program.

Preferably, the at least one web service may be able to generate a notification message, for example if a patient has forgotten to take their medicament for a predetermined time; or if it appeared that an exacerbation event may be imminent based on the patient usage data received; or if the battery associated with a medicament delivery device was running low. Such notification means are known in the prior art (eg, Mishelevich).

In one embodiment, the at least one web service may be adapted to generate a notification message itself directly to a patient or third party such as a care giver or health professional.

In another embodiment, and preferably, the at least one web service may be adapted to generate a notification message to the at least one computer program.

In such an embodiment, the at least one computer program may subsequently be adapted to generate a notification message, for example to a health professional or care giver or to the patient themselves. Suitable means for transmitting such a notification message may include SMS, a phone call or an email.

In another embodiment, the at least one web service may generate a notification to the at least one computer program, and wherein the at least one computer program may be a plurality of web services that can deliver the notification to another device associated with the same patient (for example a cell phone of the patient).

Preferably, the at least one web service may be accessible to the at least one computer program developer's software development tools for incorporation into new computer programs.

Preferably, the system may further include an access control web service for managing authentication of the at least one computer program to the at least one web service and/or authorization to use the at least one web service.

In such an embodiment, the access control web service may preferably be able to inter-operate with the at least one computer program's authentication and authorization system(s) to enable sharing of data between the at least one web service and the at least one computer program.

Preferably, the system may further include a web service which enables the at least one computer program to only access patient, patient medicament and medicament delivery device information for a provided care giver, health care professional or clinical researcher where there has already been established an association between the care giver, health care professional or clinical researcher for that patient, medicament or delivery device.

Preferably, the at least one web service may be hosted in a cloud computing infrastructure.

Alternatively, or additionally, the at least one web service may run on a number of distributed computers all located in data centres and accessible via the internet.

Alternatively, or additionally, the at least one web service may be hosted in an on-premise web hosting infrastructure.

Alternatively, or additionally, the at least one web service may exist in multiple instances.

Alternatively, or additionally, the at least one web service may exist in geographically distributed locations.

Alternatively, or additionally, the at least one web service is specified and interfaced with using standard web services protocols such as SOAP and RESTFUL

Alternatively, or additionally, the at least one web service may enable programmatic control of the behaviour of the at least one web service, such as the number of instances of the web service that should run.

It is envisaged that the at least one web service may be able to represent a virtual machine of some or all of the medicament delivery devices. For example, the virtual representation may be in a visual form depicting a picture of the medicament delivery device.

The at least one computer program may include one or more other web services.

Alternatively, or additionally, the at least one computer program may include one or more websites. In such an embodiment, the at least one web service may be in communication with the one or more websites, whereby the one or more websites may be able to request and/or receive data from the at least one web service and present the data in a human-readable format. For example, the individual or organisation responsible for managing the system may wish to view certain data (for example, relating to a particular individual or a particular patient criteria) on its own web browser. In such an embodiment, the at least one web service may be adapted to only provide the specified data to the web browser, and in a desired or requested format, for example in the form of a bar graph or table.

The at least one computer program may include a desktop application that can access the one or more web services via the internet.

The at least one computer program may include a patient management application.

The at least one computer program may include a clinical trials management application.

The at least one computer program may include a personal health software application

In the present invention, the at least one web service essentially provides a standards based means for enabling a third party's software programs to obtain and integrate the data (or the results of any operation(s) on the data) into their software programs. This is distinct from Anderson which integrates other website-based data sources (eg, a weather monitoring station) into a network computer system.

### PREFERRED EMBODIMENTS

The description of a preferred form of the invention to be provided herein, with reference to the accompanying drawings, is given purely by way of example and is not to be taken in any way as limiting the scope or extent of the invention.

### DESCRIPTION OF THE PREFERRED EMBODIMENTS

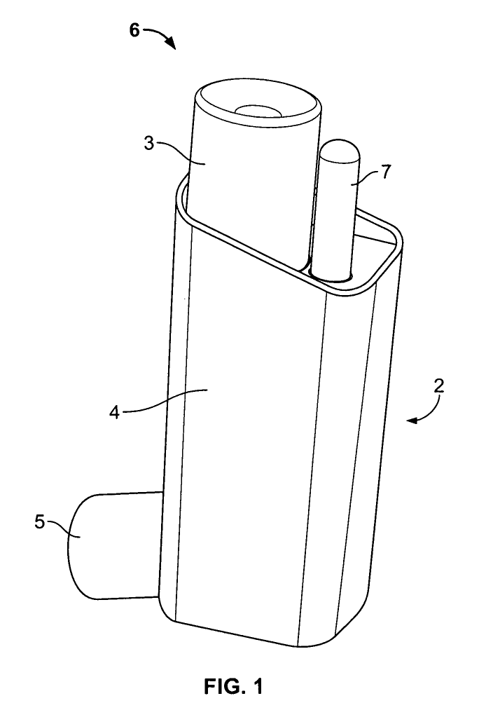

Having regard to FIG. 1, there is shown a medicament delivery device 2 in the form of a pMDI medicament inhaler, such as those used for the treatment of respiratory diseases such as asthma, COPD, cystic fibrosis, and bronchiectasis.

The medicament delivery device 2 includes a supply of medicament in the form of a medicament canister 3, which is housed within an actuator 4. The device 2 also includes a mouthpiece 5.

The medicament canister 3 includes a spray stem (not shown), which is adapted to engage with a spray-directing element (not shown) formed integrally within the actuator 4.

A metered dose of medicament may be delivered by the medicament delivery device 2 as follows:

The patient places his/her mouth over the mouthpiece 5 and pushes the medicament canister 3 downwards into the actuator 4 in the direction shown by arrow 6. This has the effect of pushing the spray stem into the spray directing element, which releases a metered dose of medicament. The metered dose of medicament is directed out of the mouthpiece 5 and into the mouth of the patient. The patient will generally inhale at the same time that the medicament is dispensed so that the medicament is inhaled as deeply as possible into the lungs of the patient.

The medicament delivery device 2 includes a controller (not shown in FIG. 1) for controlling the operation of the medicament delivery device 2 and/or for performing operation(s) on the data gathered.

The medicament delivery device 2 also includes data gathering means for gathering data relating to patient usage of the medicament delivery device 2.

The data gathering means includes an integrated electronic dose counter (not shown in FIG. 1). Each time a dose of medicament is dispensed by the device 2, the dose counter records this, along with the date and time that the dose was dispensed. The date and time is provided by a real time clock (not shown in FIG. 1) incorporated within the electronic dose counter and/or the controller.

The data gathered is stored within a memory (not shown in FIG. 1) associated with the device 2. It is also envisaged that other data relating to patient usage of the device 2 may also be recorded or monitored, for example inhalation data

The device 2 also includes a user interface (not shown in FIG. 1) which allows the patient to access data recorded or received by the device 2, and also to change the settings of the device 2. The user interface includes operational buttons and a LCD screen.

The device 2 includes a power management system (not shown in FIG. 1), for example a battery. The battery may be a replaceable battery or a rechargeable battery.

The controller includes a microprocessor (not shown in FIG. 1) for performing operations on the data thus gathered. Furthermore, the controller is also able to actuate (or deactivate) components of the medicament delivery device 2, based on data or instructions received by the medicament delivery device 2. For example, the medicament delivery device 2 may receive instructions to cease the dispensing of doses for a limited time and/or to increase or decrease the amount of medicament dispensed during each dose.

The real time clock is able to note the date and time associated with each dose of medicament dispensed by the device 2, as well as being able to note the date and time of any other event, for example when a new canister of medicament is placed within the device 2, or when the battery runs out or is getting low, or when instructions have been received by the medicament delivery device 2 to cease the ability to dispense doses, and so on.

The device 2 also includes an integrated wireless data communication means, generally indicated by arrow 7. The wireless data communicator 7 is able to transmit data gathered by the device 2 to at least one web service, and preferably to a communications web service which comprises part of the at least one web service. The wireless data communicator 7 may be adapted to transmit the data in real time or preferably at pre determined intervals, for example one or twice a day or after a predetermined number of inhalations by the patient.

The wireless data communicator 7 includes a cell phone chip (not shown in FIG. 1) housed within the device 2.

Such compliance monitoring means for medicament delivery devices 2 (as described in very general terms above) are well known in the prior art and to those skilled in the art, and it is not considered necessary therefore to describe their operations in any significant further detail herein.

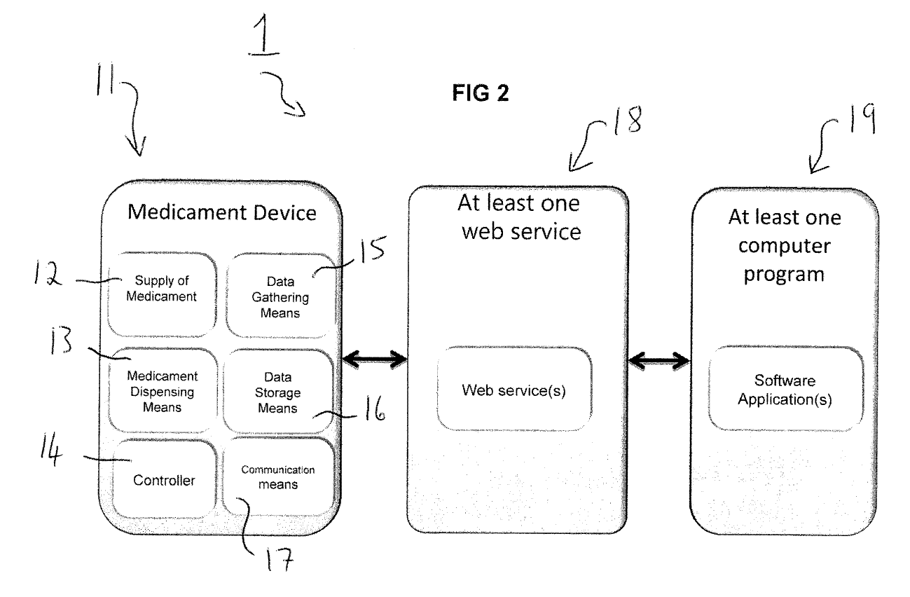

Having regard to FIG. 2 there is shown a system, generally indicated by arrow 1, for managing data relating to patient usage of a medicament delivery device 11.

The schematically-represented system 1 includes the following features or components:

The medicament delivery device 11 includes a supply of medicament 12 and a medicament dispensing means 13. The medicament delivery device 11 further includes a controller 14, a data gathering means 15, and a data storage means 16,
The device 11 also includes a wireless communications means 17 in the form of a wireless transmitter for the transmission of data to at least one web service, generally indicated by arrow 18.

The at least one web service 18 is able to share any of the data with at least one computer program, generally indicated by arrow 19. The at least one computer program 19 may be a third party's software program(s) which is able to obtain and integrate the data (or the results of any operation(s) on the data) into their software programs.

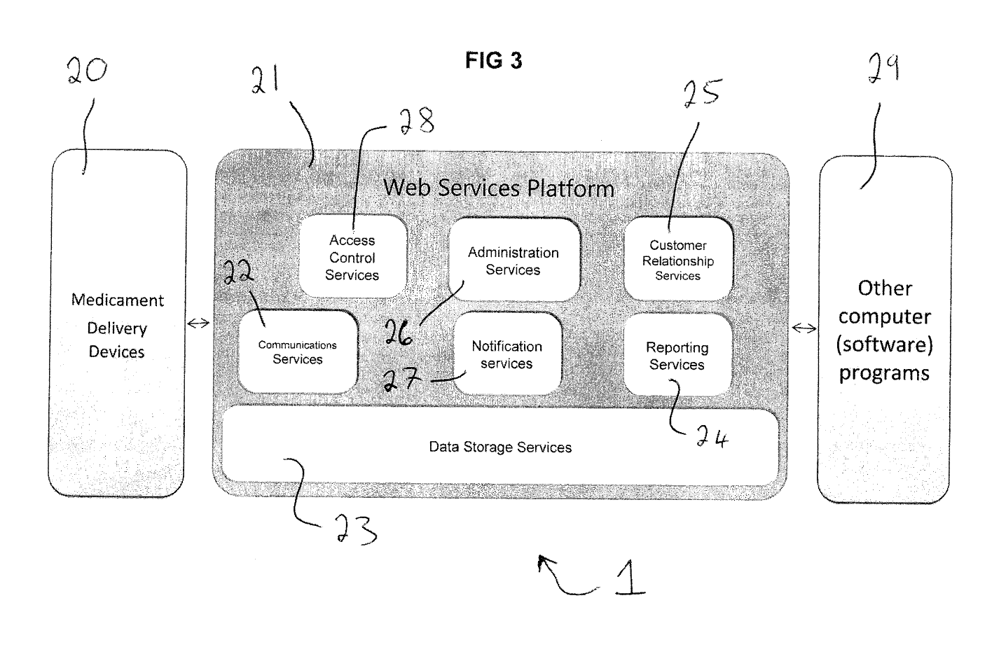

Having regard to FIG. 3, there is shown an expanded view of the at least one web service 18 depicted in FIG. 2.

In FIG. 3 there is represented a plurality of medicament delivery devices 20 and at least one web service represented by the web services platform 21. The web services platform 21 includes a communications web service(s) 22, a data storage web service(s) 23, a reporting web service(s) 24, a customer relationship web service(s) 25, an administration web service(s) 26, a notification web service(s) 27 and an access control web service(s) 28.

The web services platform 21 is able to share data with a number of other (third parties') computer programs 29.

The communications web service 22 effectively acts as the web service platform's communication interface with the medicament delivery device 20, and in particular with the communications means associated with the medicament delivery device 20. The communications web service 22 is able to both send and receive data or information or instructions with respect to the medicament delivery device 20.

Any data sent from the medicament delivery device 20 to the web services platform 21, via the communications web service 22, is forwarded to the data storage services 23, to be stored therein.

The medicament delivery device 20 is able to send any data gathered by and/or stored within the medicament delivery device 20. For example, the medicament delivery device 20 may able to transmit data relating to settings of the medicament delivery device 20 and/or data relating to a patient's prescribed treatment regimen and/or data relating to diagnostic information concerning operation of the medicament delivery device 20, and so on. Further examples of the type of data that may be gathered by and/or transmitted from the medicament delivery device 20 have been previously described in this specification.

The web services platform 21 is able to receive, queue and/or store information received from the other computer programs 29, for immediate or scheduled transmission to the medicament delivery device 20.

For example, information received from the other computer programs 29 may include data relating to settings of the medicament delivery device 20 and/or data relating to a patient's prescribed treatment regimen and/or data relating to the operation of the medicament delivery device 20. Other examples of data or information that may be received by the other computer programs 29 have been provided previously in this specification. Any such data may preferably be stored in the data storage web service 23.

The reporting web service 24 may be able to perform processes and/or computations upon the data gathered, in order to produce a report, with the report being deliverable to the other computer programs 29 upon request.

Any type of processes or computations upon the data may be carried out, as required or as desired, or as dictated by the type of patient compliance monitoring being undertaken. Examples of the types of processes and/or computations that may be undertaken on the data gathered have been previously described in this specification.

The customer relationship web service 25 enables another computer program 29 to add, edit and/or delete data stored within said data storage web service 23. Examples of the types of additions, edits or deletions that may be undertaken on the data have been previously described in this specification.

The administration web service 26 can create, update and/or remove accounts for the other computer programs 29 which access and/or utilize the web services platform 21.

For example, the administration web service 26 may transact payment and billing services in exchange for access to the web services platform 21 by the other computer programs 29. Examples of other types of functions undertaken by the administration web service 26 have been previously described in this specification.

The notification web service is able to generate a notification message to the other computer programs 29, for example a notification alerting the programs 29 that a patient has forgotten to take their medicament for a predetermined time period, or if it appears that an exacerbation event may be imminent based on the patient usage data received.

In such an embodiment, the other computer programs 29 may be adapted to generate a notification message, for example to a health professional or care giver or the patient themselves. Suitable means for transmitting such a notification message may include SMS, a phone call or an email.

The access control web service 28 is adapted to manage authentication of the other computer programs 29 to the web services platform 21 and/or authorization of the other computer programs 29 to use the web services platform 21. In such an embodiment, the web services platform 21 may be able to federate with the other computer program's authentication systems to let claims created in one identity scope be accepted by another.

The access control web service 28 also includes a web service which enables other computer programs 29 to only access patient, patient medicament and medicament delivery device information for a provided care giver, health care professional or clinical researcher where there has already been established an association between the care giver, health care professional or clinical researcher for that patient, medicament or delivery device.

Examples of suitable computer programs 29 for use with the system 1 are a health information system and a clinical trials application. Such programs 29 may be owned or run by third party vendors who have an interest in monitoring the patient data gathered and who therefore pay for the privilege of being able to access data via the web services platform 21 (eg, the vendors may be insurance companies checking to ensure their customers are complying with their medicament as per the insurance agreement between them; or the vendors may be medical professionals monitoring patient usage data for any events which may be indicative of non-compliance or an exacerbation event).

Each such software program 29 is able to request specific data from the web services platform 21, and the web services platform 21 is subsequently able to send the data (usually in a format defined by the web services platform 21).

For example, a health information program may request data from the web services platform 21 relating to all their customers who have missed at least one scheduled dose of a preventer medicament in the last 48 hours. The web services platform will then supply this information in a predetermined format. This request may be handled, for example, by a first reporting web service 24. Simultaneously, a second reporting web service 24 may be responding to a request from a clinical trials program relating to the number of trialists who are overusing their medicament, and this response may thus be transmitted to the clinical trials program.

The web services platform 21 may be hosted in a cloud computing infrastructure. Examples of possible make ups of such a cloud computing infrastructure have been previously provided in this specification.

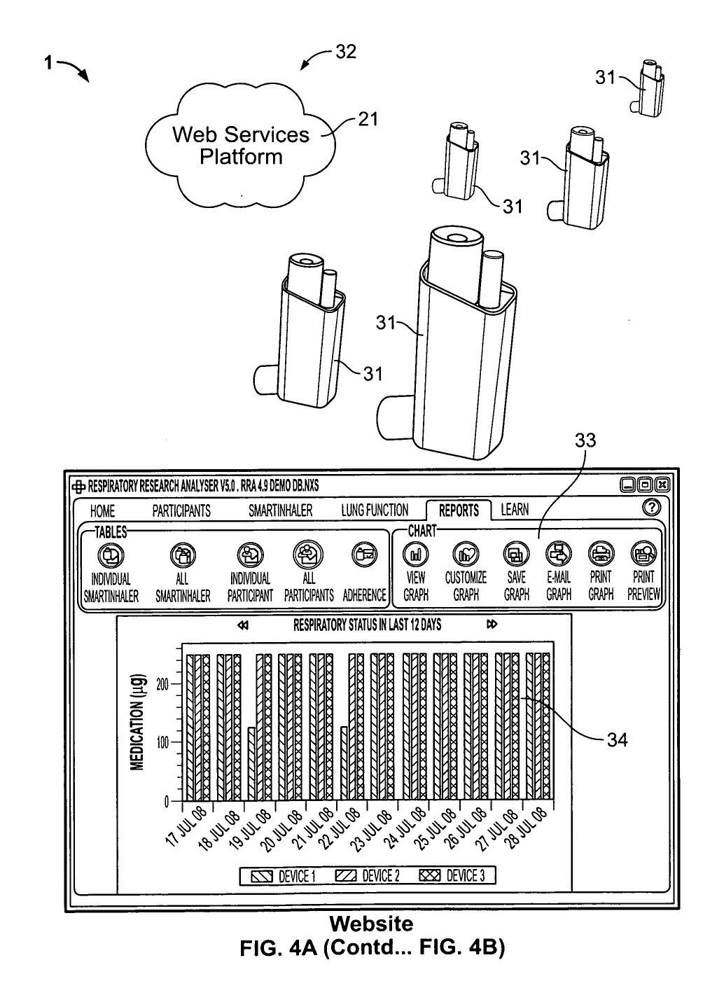

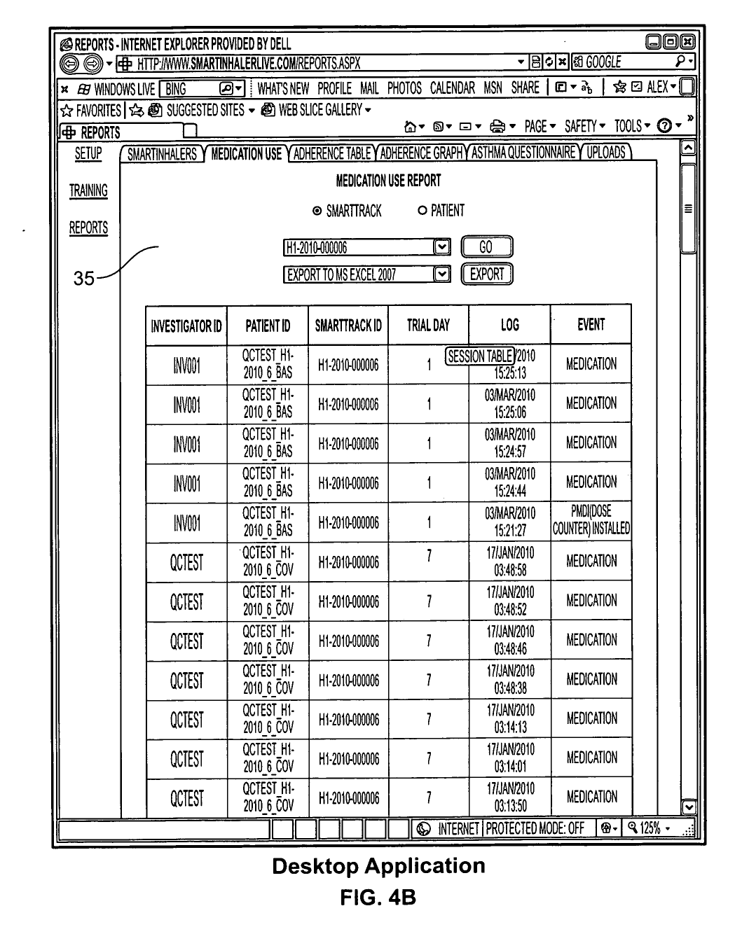

FIG. 4 illustrates how such a cloud computing infrastructure may be represented. The web services platform 21 is hosted in a cloud computing infrastructure 32 as shown. A plurality of medicament delivery devices 31 are in communication with the cloud computing infrastructure 32.

Furthermore, there is shown a computer program in the form of a web site 33 which is also in communication with the cloud computing infrastructure 32.

The website 33 is able to request and receive data from the web services platform 21 and present the data in a human-readable format (for example, in the form of a bar graph 34). For example, the individual or organisation responsible for the website 33 may wish to view certain data (for example, relating to the number of doses of medicament a patient takes over a 20 day period) on its own web browser—of which the website 33 is operatively connected or comprises part of. In such an embodiment, the web services platform 21, housed within the cloud computing infrastructure 32, may be adapted to provide the specified data to the web browser (and therefore the website 33) in the desired or requested format.

FIG. 4 also shows a desktop application 35 which can similarly access the web services platform 21 via the internet.

An advantage of the system 1 over and above prior art patient compliance monitoring systems (which generally use integrated data sources connected to a network computer system—eg Anderson) is that the system 1 provides for a standards based means for enabling other vendor's software programs 29 to obtain and integrate medicament delivery device information into their own software programs.

The invention may be useful for use with large clinical trials, for example those used to determine whether a particular medicament may have unwanted side effects. Such trials may include a number of people in a trial group (those using the medicament being tested) and a number of people in the control group (those not using the medicament being tested). There may be upwards of 3000 people in each group, and the results of the clinical trail may be very important. Hence, it is imperative that the people running the trial have ready access to the data from the trial, and in a useful format. Moreover, it would be of advantage if the people running the trial were able to integrate patient usage data relating to use of the medicament device into their own software programs. The system 1 allows for this.

### VARIATIONS

While the embodiments described above are currently preferred, it will be appreciated that a wide range of other variations might also be made within the general spirit and scope of the invention and/or as defined by the appended claims.

We claim:

1. A system for managing data relating to patient usage of a medicament delivery device, said medicament delivery device including:
a) a supply of medicament,
b) a medicament dispensing means,
c) data gathering means for gathering data relating to patient usage of said medicament delivery device,
d) a data storage means for storing said data, and
e) communication means for transmitting said data to at least one web service,
and wherein said at least one web service is able to share said data with at least one computer program.

2. A system as claimed in claim 1, wherein said at least one web service includes a communications web service for communicating with said communication means.

3. A system as claimed in claim 1, wherein said at least one web service includes a data storage web service for storing data transmitted from said medicament delivery device.

4. A system as claimed in claim 1, wherein said medicament delivery device further includes a controller for controlling the operation of said medicament delivery device and/or for performing operation(s) on said data.

5. A system as claimed in claim 4, wherein said controller includes a micro processor for performing operations on said data.

6. A system as claimed in claim 4, wherein said controller is able to control the operation of said medicament dispensing means.

7. A system as claimed in claim 1, wherein said data gathering means includes a dose counter, said dose counter being able to determine when a dose of medicament has been dispensed by said medicament delivery device.

8. A system as claimed in claim 1, wherein said communication means transmits data to said at least one web service using wireless communication means.

9. A system as claimed in claim 1, wherein said medicament delivery device is adapted to retain a copy of any data transmitted to said at least one web service in said data storage means.

10. A system as claimed in claim 1, wherein said medicament delivery device is adapted to transmit any data to said at least one web service, without retaining a copy of said data in said data storage means.

11. A system as claimed in claim 1, wherein said medicament delivery device is able to transmit, to said at least one web service, data relating to settings of the medicament delivery device and/or data relating to a patient's prescribed treatment regimen and/or data relating to diagnostic information concerning operation of the medicament delivery device.

12. A system as claimed in claim 1, wherein said at least one web service is also able to receive information or data from said at least one computer program.

13. A system as claimed in claim 12, wherein said information received from said at least one computer program is received, queued and/or stored within a data storage web service.

14. A system as claimed in claim 12 wherein said information received from said at least one computer program includes data relating to settings of the medicament delivery device and/or data relating to a patient's prescribed treatment regimen and/or data relating to the operation of said medicament delivery device.

15. A system as claimed in claim 14, wherein said information received from said at least one computer program includes software for execution on said medicament delivery device.

16. A system as claimed in claim 1, wherein said at least one web service is able to perform processes and/or computations upon said data, in order to produce a report, said report being deliverable to said at least one computer program upon request.

17. A system as claimed in claim 16, wherein said at least one web service provides, to said at least one computer program, a dataset which includes details of patients and/or details of medicament delivery devices and/or details of medicaments associated with each patient.

18. A system as claimed in claim 3, wherein said system includes a further web service that enables another computer program to add, edit and/or delete data stored within said data storage web service.

19. A system as claimed in claim 1, wherein said system includes one or more system administration web services that can create, update and/or remove accounts for said at least one computer program which accesses and/or utilizes said at least one web service.

20. A system as claimed in claim 1, wherein said system includes one or more web services that transact payment and billing services in exchange for access to said at least one web service by said at least one computer program.

21. A system as claimed in claim 1, wherein said at least one web service can generate a notification message.

22. A system as claimed in claim 21, wherein said at least one web service can generate a notification message to said at least one computer program.

23. A system as claimed in claim 21, wherein said at least one web service is adapted to generate a notification when a patient's medicament usage patterns are in a manner which is consistent with a developing exacerbation.

24. A system as claimed in claim 21 wherein said at least one computer program includes a plurality of web services that can deliver the notification to another device associated with the same patient.

25. A system as claimed in claim 1 wherein said at least one web service is accessible by said at least one computer program developer's software development tools for incorporation into a new computer program(s).

26. A system as claimed in claim 1 wherein said system further includes an access control web service for managing authentication of said at least one computer program to said at least one web service and/or authorization for said at least one computer program to use said at least one web service.

27. A system as claimed in claim 26 wherein said access control web service can inter-operate with said at least one computer program's authentication and authorization system(s) to enable sharing of data between said at least one web service and said at least one computer program.

28. A system as claimed in claim 1, wherein the system includes a web service which enables said at least one computer program to only access patient, medicament. and medicament delivery device information for a provided care giver, health care professional or clinical researcher where there has already been established an association between the care giver, health care professional or clinical researcher for that patient, medicament or medicament delivery device.

29. A system as claimed in claim 1, wherein said at least one web service is hosted in a cloud computing infrastructure.

30. A system as claimed in claim 1 wherein said at least one web service runs on a number of distributed computers all located in data centres and accessible via the internet.

31. A system as claimed in claim 1 wherein said at least one web service is hosted in an on-premise web hosting infrastructure.

32. A system as claimed in claim 1 wherein said at least one web service can exist in one or more instances.

33. A system as claimed in claim 1 wherein said at least one web service can exist in geographically distributed locations.

34. A system as claimed in claim 1 wherein said at least one web service is specified and interfaced using standard web services protocols such as SOAP and RESTFUL.

35. A system as claimed in claim 1 wherein said at least one web service enables programmatic control of the behaviour of said at least one web service such as the number of instances of said at least one web service that should run.

36. A system as claimed in claim 1, wherein said at least one web service represents a virtual machine of one or more of said medicament delivery devices.

37. A system as claimed in claim 1 wherein said at least one computer program includes one or more other web services.

38. A system as claimed in claim 1 wherein said at least one computer program includes one or more web sites.

39. A system as claimed in claim 1 wherein said at least one computer program includes a desktop application that can access said at least one web service via the internet.

40. A system as claimed in claim 1 wherein said at least one computer program includes a patient management application.

41. A system as claimed in claim 1 wherein said at least one computer program includes a clinical trials management application.

42. A system as claimed in claim 1 wherein said at least one computer program includes a personal health software application.

43. A system as claimed in claim 1, wherein said medicament delivery device includes at least one medicament inhaler.
